(19) United States
(12) Patent Application Publication
(10) Pub. No.: US 2026/0033944 A1
(43) Pub. Date: Feb. 5, 2026

(54) NASAL DEVICE AND METHODS OF USE AND MANUFACTURE

(71) Applicant: Gregory A. Hogle, Denver, CO (US)

(72) Inventor: Gregory A. Hogle, Denver, CO (US)

(21) Appl. No.: 19/284,154

(22) Filed: Jul. 29, 2025

Related U.S. Application Data
(60) Provisional application No. 63/677,929, filed on Jul. 31, 2024.

(51) Int. Cl. A61F 2/18

(52) U.S. Cl. CPC A61F 2/186; A61F 2230/0006; A61F 2250/0037

(57) ABSTRACT

A septum support or nasal splint that is insertable within a nasal cavity of a patient to maintain a position of a septum of the patient includes a base and a first side member that is positioned on a first side of the base and that extends upward from the base. The first side member is configured to contact the septum of the nasal cavity. The septum support also includes a second side member that is positioned on a second side of the base and that extends upward from the base. The second side member is configured to contact a turbinate of the nasal cavity so as to reinforce the first side member. A lower end of the second side member is securely connected to the base or first side member and an upper end of the second side member is not attached to the first side member.

### CROSS-REFERENCE TO RELATED APPLICATION

This application claims the benefit of and priority to U.S. Provisional Application No. 63/677,929, filed on Jul. 31, 2024, and titled “NASAL DEVICE AND METHODS OF USE AND MANUFACTURE,” the content of which is herein incorporated by reference in its entirety for all purposes.

### TECHNOLOGICAL FIELD

The subject invention relates generally to nasal devices and methods of using and manufacturing the same. More particularly, nasal splints are described herein that are designed for supporting and stabilizing the nasal septum and associated anatomical structures following surgical intervention.

### BACKGROUND

Various nasal surgeries may be performed to treat a variety of issues. For example, septoplasty may be performed to correct a deviated septum. To maintain the corrected, straight position of the septum during recovery, splints, such as a pair of Doyle splints, can be placed at either side of septum until the septum heals.

Other common nasal surgeries may include sinus surgery, which may be performed to open the pathways of the sinuses and clear blockages. During the sinus surgery, the middle turbinate, which tends to impede access to the sinuses, must be displaced in order for the surgeon to gain access to the sinus cavity. Techniques for displacing the middle turbinate include using a surgical instrument, such as a Freer elevator, to compress the middle turbinate towards the septum. Due to the middle turbinate's tendency to migrate laterally, or toward the side wall of the nose, repeat compressions of the middle turbinate may be necessary during the surgery.

### BRIEF SUMMARY

The embodiments described herein provide nasal devices, such as septum support and/or nasal stents, having improved properties over conventional nasal devices. According to one aspect, a nasal splint that is configured for insertion into a patient's nasal cavity to stabilize a septum includes a base, a septum support situated on a first side of the base and extending upward therefrom, and a side support located on a second side of the base and extending upward therefrom. The septum support is configured to engage the septum within the nasal cavity and the side support is configured to contact a turbinate of the nasal cavity to reinforce the septum support. A lower end of the side support is securely connected to either the base or septum support and an upper end of the side support is unattached to the septum support. The side support has an arcuate or semicircular profile such that its lower end curves outward away from the septum support and its upper end curves inward toward the septum support.

The side support extends upward from the base to a height that is less than half a height of the septum support as measured from the base. In such a configuration, the side support does not contact a middle turbinate of the nasal cavity. In a specific instance, the side support extends upward from the base between 6 and 8 mm and the septum support extends upward from the base between 20 and 25 mm. The septum support may include one or more reinforcing ribs that extend longitudinally along a length of the septum support and that protrude laterally from a surface of the septum support in a direction toward the side support. In such instances, at least one reinforcing rib may be aligned with a top edge of the side support.

The base, the septum support, and the side support may define or form a curved channel or trough. Opposing ends of the base may curve downward relative to a straight central section and/or at least one end of the base may include an aperture to allow the nasal splint to be anchored to the septum using a suture. The side support may include a groove that extends along a longitudinal length of the side support.

According to another aspect, a nasal splint includes a base, a septal plate or lamella positioned on a first side of the base and extending upward therefrom, and a turbinate brace positioned on a second side of the base and extending upward therefrom. A lower end of the turbinate brace may be securely connected to the base or to the septal plate or lamella and an upper end of the turbinate brace may not be attached to the septal plate or lamella. The turbinate brace may have an arcuate or semicircular profile such that the lower end of the turbinate brace curves outward away from the septal plate or lamella and the upper end of the turbinate brace curves inward toward the septal plate or lamella.

The turbinate brace may extend upward to a height that is less than half a height of the septal plate or lamella as measured from the base such that turbinate brace does not contact a middle turbinate. In a specific embodiment, the turbinate brace may extend upward from the base between 6 and 8 mm and the septal plate or lamella may extend upward from the base between 20 and 25 mm. The septal plate or lamella may include one or more reinforcing ribs that extend longitudinally along a length of the septal plate or lamella and that protrude laterally from a surface of the septal plate or lamella. In such instances, at least one reinforcing rib may be aligned with a top edge of the turbinate brace. The base, septal plate or lamella, and turbinate brace nay define or form a curved channel or trough. Opposing ends of the base may curve downward relative to a straight central section. The turbinate brace may include a groove that extends along a longitudinal length of the turbinate brace.

According to another aspect, a method for supporting a septum of a nasal cavity includes inserting a nasal splint into one side of the nasal cavity, in which the nasal splint includes a base, a septal plate or lamella, and a turbinate brace. The method also includes placing the septal plate or lamella into contact with the septum and placing the turbinate brace into contact with a turbinate of the nasal cavity so that the turbinate brace engages the turbinate. A lower end of the turbinate brace is securely connected to the base or to the septal plate or lamella and an upper end of the turbinate brace is not attached to the septal plate or lamella. The septal plate or lamella and the turbinate brace are biased away from one another such that pressure is applied to the septum by the septal plate or lamella.

The method may further include attaching the septal plate or lamella to the septum to maintain a position of the nasal splint relative to the septum and/or grasping the nasal splint by positioning a blade or jaw of a forcep within a groove that extends along a longitudinal length of the turbinate brace.

### BRIEF DESCRIPTION OF THE DRAWINGS

The present invention is described in conjunction with the appended figures:

FIG. 1 illustrates a cross sectional view of part of the head along the parasagittal plane.

FIG. 2A illustrates a side or lateral view of an embodiment of a septum support that is configured for retaining a breathing passage and/or for maintaining a position of a septum of a patient.

FIG. 2B illustrates a front or anterior view of the septum support of FIG. 2A.

FIG. 2C illustrates a front or anterior view of another embodiment of a septum support.

FIG. 2D illustrates a septum support having a reinforcing member positioned at a first location.

FIG. 2E illustrates a septum support having a reinforcing member positioned at various locations.

FIG. 2F illustrates a side or lateral view of another embodiment of a septum support that is configured for retaining a breathing passage and/or for maintaining a position of a septum of a patient.

FIG. 2G illustrates a front or anterior view of the septum support of FIG. 2F.

FIG. 3A illustrates a front or anterior view of an embodiment of a nasal splint or septum support that is configured for retaining a breathing passage and/or for maintaining a position of a septum of a patient.

FIG. 3B illustrates a front or anterior view of another embodiment of a nasal splint or septum support that is configured for retaining a breathing passage and/or for maintaining a position of a septum of a patient.

FIG. 3C illustrates a side or lateral view of the nasal splint or septum support of FIG. 3A or 3B.

FIG. 3D illustrates a front or anterior view of another embodiment of a nasal splint or septum support that is configured for retaining a breathing passage and/or for maintaining a position of a septum of a patient.

FIG. 3E illustrates a side or lateral view of the nasal splint or septum support of FIG. 3D.

FIG. 4 illustrates a front or anterior view of a pair of septum supports that operate together to maintain an upright position of the septum.

FIG. 5 is a simplified flow chart illustrating a method of using septum supports described herein to help retain a breathing passage and/or to maintain the position of the septum for a patient.

FIG. 6 is a simplified flow chart illustrating a method of using septum supports described herein to help retain a breathing passage and/or to maintain the position of the septum for a patient.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION

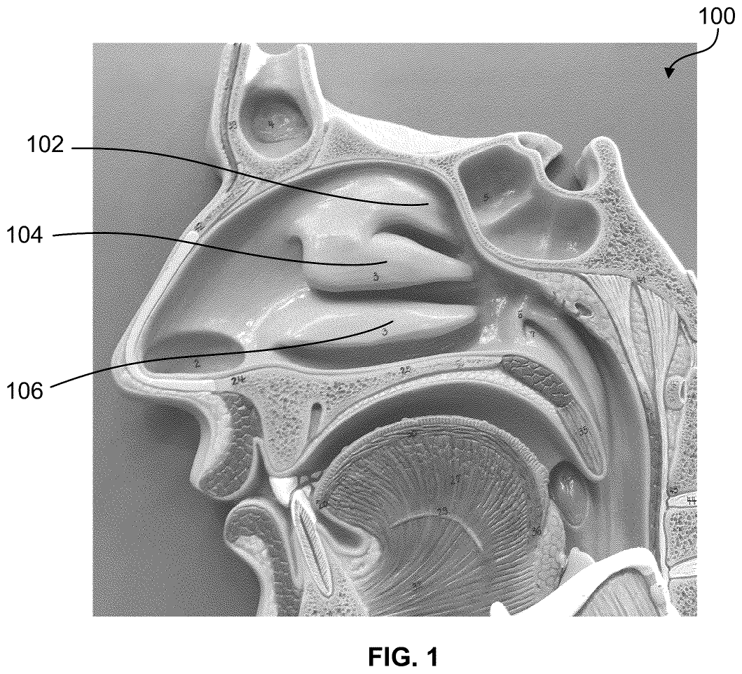

The nasal path is typically a curvy cavity or channel within the head. Several turbinates (a boney framework covered by soft spongy tissue) are positioned within the nasal cavity. FIG. 1 illustrates a cross sectional view 100 of part of the head along a parasagittal plane. The septum is removed from FIG. 1 to better illustrate the details inside the nasal cavity. There are most commonly three turbinates inside the nasal cavity: an upper or superior turbinate 102, a middle turbinate 104, and an inferior or lower turbinate 106. The turbinates cause laminar airflow (a physiologically important function) through the nose.

The septum, which separates the left and right airways of the nasal cavity, is commonly deviated or crooked. Operational procedures, such as septoplasty, may be performed to correct a deviated septum. During recovery after an operational procedure, the septum may benefit from support so as to maintain and heal in a corrected, straight position.

Existing techniques for maintaining the corrected, straight position of the septum include placing splints, such as a pair of Doyle splints, on either side of the septum until the septum heals in the corrected, straight position. A Doyle splint includes a planar piece, one side of which is configured to contact one side of the septum and the other side of which is integrally formed with a curved piece to form an elongated half pipe. The planar pieces of a pair of the Doyle splints, disposed at either side of the septum, cooperate to maintain the straight position of the septum, and the half pipes of the Doyle splints operate as breathing passages for the patient.

One issue commonly encountered by patients with the Doyle splints is that the half pipes tend to get clogged, given its elongated shape, and fails to function as a breathing passage. About 50% of the surgeons avoid using the Doyle splints due to such blockage. Additionally, the curved piece extends along substantially the entire length of the planar piece. When the Doyle splint is placed into the nasal cavity, the posterior aspect of the curved half pipe component compresses the middle turbinate 104 laterally. The term “laterally” means outward from the midline of the body, and the term “lateral” is used herein to describe a position that is further from the midline of the body. If sinus surgery has been performed in combination with septoplasty, then postoperative healing for the sinus surgery component may be compromised by the half pipe component causing lateralizing compression of the middle turbinate 104. This problem is another reason that surgeons choose not to insert the Doyle splints.

In some aspects, the present technology described herein provides a septum support configured to maintain the septum in an upright or straight position while avoiding at least some or all of the issues discussed above. In some embodiments, a pair of the septum supports as described herein may be used to support the septum and to provide a breathing passage. Each septum support may include a base member, a first side member, and a second side member that collectively define a channel or a trough with uneven sides, such as the U-shaped, C-shaped, or curved channel described and illustrated herein. As compared to the half-pipe design of the Doyle splint, the more open design of the septum support provides a breathing passage for the patient that is less likely to be blocked, and any accumulation can be easily rinsed out.

The first side member may be characterized by a height and a length that substantially corresponds to the height and the length of the septum, respectively, such that the first side member supports the entire septum vertically and longitudinally. The second side member may be characterized by a height less than that of the first side member such that it contacts the inferior or lower turbinate while limiting or avoiding contact with the middle turbinate. Additionally, the second side member includes a length that is less than the length of the first side member such that the second side member does not extend as far back into the nasal cavity as the first side member, not only avoiding contact with the middle turbinate, but also preventing blockage or irritation to the sinuses.

In some embodiments, the second side member further includes a curved portion that defines a convex or concave surface. The concave surface may substantially conform to the curvature or contour of the inferior or lower turbinate. As compared to the conventional Doyle splint which contacts the surfaces of one or more turbinates with the convex surface of its half pipe, the concave surface of the second side member of the present technology distributes the pressure imparted onto lateral nasal tissue over a smaller surface area, and thus is more comfortable for the patient. In embodiments with a convex surface, the second side member may have a bottom portion that curves away from the first side member and a top portion that curves back toward the first side member. In such embodiments, the convex surface may be shaped and sized so that it is positioned under the middle turbinate rather than contacting the middle turbinate.

The base member may have a planar design, which provides a greater surface area for the septum support to rest on the floor of the nasal cavity. In contrast, the Doyle splint rests on the bottom edge of its planar piece when placed in the nasal cavity, which can cause discomfort and/or irritation to the nose. The planar design of the base member of the nasal support described herein significantly reduces or limits such discomfort and/or irritation.

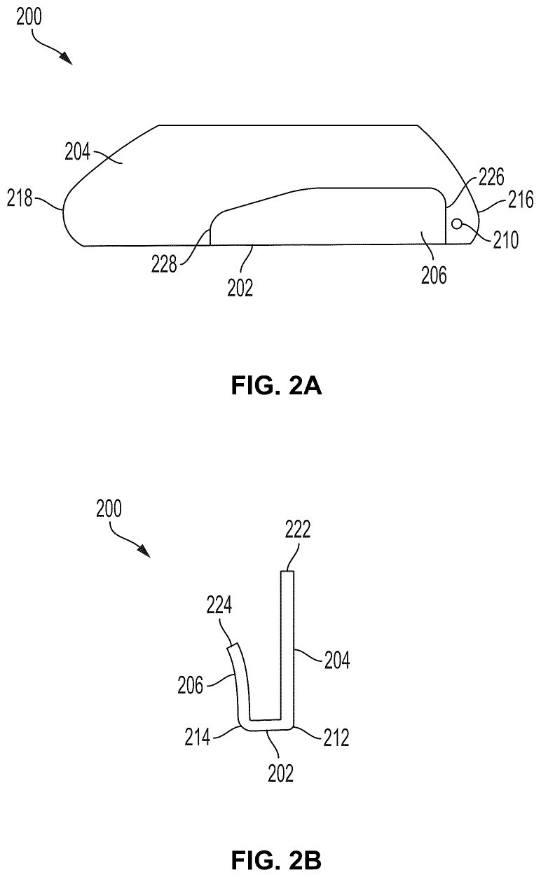

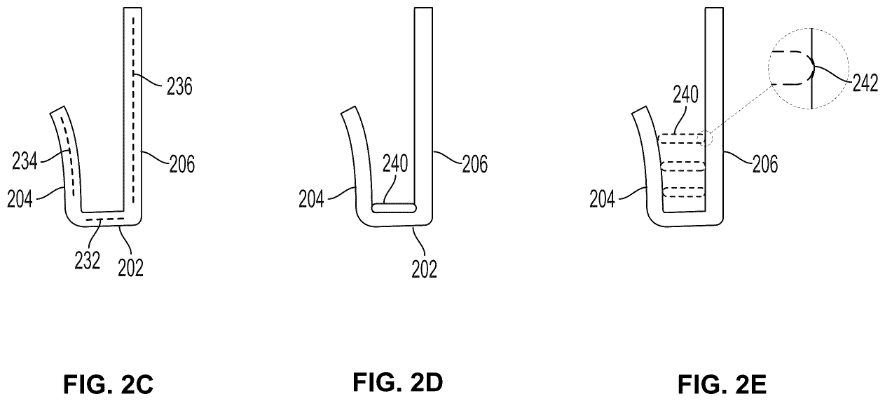

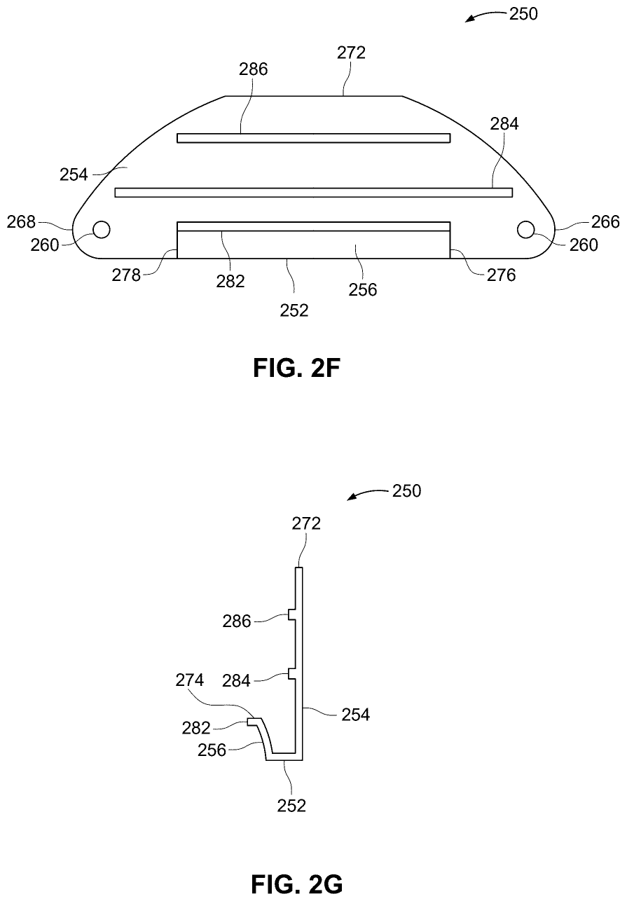

FIG. 2A illustrates a side or lateral view of a nasal splint or septum support 200 that is configured for retaining a breathing passage and/or for maintaining a position of a septum of a patient. FIG. 2B illustrates a front or anterior view of the septum support 200. The septum support 200 illustrated in FIGS. 2A and 2B is a right septum support that is configured to be placed or inserted into the right side of the nasal cavity. As will be described in more detail below, in operation, the right septum support operates together with a left septum support that is constructed similarly to the right septum support but is configured to be placed or inserted into the left side of the nasal cavity to maintain the septum in an upright or straight position and/or to help retain a breathing passage for the patient.

As shown in FIGS. 2A and 2B, the septum support 200 includes a base member 202, a first side member 204 extending upward away from a first edge 212 of the base member 202, and a second side member 206 extending upward away from a second edge 214 of the base member 202. The base member 202, the first side member 204, and the second side member 206 collectively define a U-shaped channel or a trough with uneven sides. In other words, the first side member 204 and the second side member 206 define an open end opposite to the base member 202 joining the first side member 204 and the second side member 206. Compared to the half-pipe design of a Doyle splint, the open design of the septum support 200 provides a breathing passage for the patient that is less likely to be blocked, and any accumulation can be rinsed out with saline or other solutions.

The base member 202 is substantially planar and is configured to rest on a bottom or floor of the nasal cavity of the patient. The first and second edges 212, 214 of the base member 202 define a width of the base member 202 that helps to maintain the orientation and/or position of the septum support 200 in the nasal cavity, including maintaining a generally upright position of the first side member 204 so as to keep the septum straight. In some embodiments, the width of the base member 202 ranges between about 3 mm and about 8 mm, between about 3 mm and about 7 mm, or between about 4 mm and about 6 mm, such as about 4 mm. The width of the base member 202 may be consistent throughout its longitudinal extension. The planar design of the base member 202 provides a greater surface area for the septum support 200 to rest on the floor of the nasal cavity as compared to the existing technologies. For example, in a conventional Doyle splint, only the bottom edge of the planar piece of the Doyle splint may rest on the floor of the nasal cavity, which can cause discomfort and/or irritation to the nose. The planar base member 202 of the present technology significantly reduces or limits such discomfort and/or irritation.

As will be described in more detail below, the first side member 204 and the second side member 206 have different lengths. In some embodiments, the base member 202 may include a length that is substantially the same as the length of the first side member 204. In other words, the base member 202 and the first side member 204 may be coextensive along the longitudinal dimension of the septum support 200. In some embodiments, the base member 202 may include a length that is substantially the same as the length of the second side member 206. That is, the base member 202 and the second side member 206 may be coextensive along the longitudinal dimension of the septum support 200. In some embodiments, the base member 202 may include tapered end portion or portions that expand between the longitudinal ends of the first side member 204 and the second side member 206.

As mentioned above, the septum support 200 illustrated in FIGS. 2A and 2B is a right septum support configured to be placed on the right side of the nasal cavity. When the septum support 200 is inserted into the right side of the nasal cavity, the first side member 204 is configured to contact the right side of the septum, and the second side member 206 is configured to contact a turbinate, such as the inferior or lower turbinate 106, on the right lateral wall of the nasal cavity.

The first side member 204 is substantially planar so as to maintain the septum in a substantially upright or straight position. The first side member 204 includes a length defined by a proximal edge 216 and a distal edge 218 of the first side member 204. The length of the first side member 204 may substantially correspond to the length of the septum such that the first side member 204 supports substantially the entire septum along the longitudinal extension of the septum. In some embodiments, the length of the first side member 204 ranges between about 65 mm and about 75 mm, or between about 65 mm and about 70 mm, such as about 68 mm. The first side member 204 includes a height defined by an upper edge 222 of the first side member 204 and the first edge 212 of the base member 202 such that the first side member 204 supports substantially the entire septum vertically. In some embodiments, the height of the first side member 204 ranges between about 15 mm and about 25 mm, between about 17 mm and about 23 mm, or between about 19 mm and about 23 mm, such as about 21 mm. The upper edge 222 of the first side member 204 may be substantially straight while the proximal and distal edges 216, 218 may be curved so as to form tapered proximal and distal ends of the septum support 200 to facilitate the insertion and removal of the septum support 200 into and out of the nasal cavity.

In some embodiments, the first side member 204 includes an aperture or suture hole 210 near the proximal edge 216 of the first side member 204 so that the first side member 204 can be sutured to the septum near the columella, which is the soft tissue and skin overlying the anterior edge of the septum, through the suture hole 210 to further secure the position of the septum support 200 inside the nasal cavity. In some embodiments, the first side member 204 may be adhered to the septum support 200 using adhesive or glue as an alternative to or in addition to suturing.

As mentioned above, the second side member 206 is configured to contact a turbinate on the right lateral wall of the nasal cavity. More specifically, the second side member 206 is configured to contact the inferior or lower turbinate 106 while limiting or avoiding contact with the middle turbinate 104. Thus, the second side member 206 includes a height as defined by an upper edge 224 of the second side member 206 and the second edge 214 of the base member 202 that is less than the height of the first side member 204 so as to limit or avoid contact with the middle turbinate 104. Additionally, the second side member 206 includes a length as defined by a proximal edge 226 and a distal edge 228 of the second side member 206 that is less than the length of the first side member 204 such that the second side member 206 does not extend as far back into the nasal cavity as the first side member 204 so as to limit or avoid contact with the middle turbinate 104, as well as to limit or prevent blockage or irritation to the sinuses. In some embodiments, the height of the second side member 206 ranges between about 3 mm and about 13 mm, or between about 4 mm and about 8 mm, such as about 5 mm. In some embodiments, the length of the second side member 206 ranges between about 35 mm and about 45 mm, or between about 37 mm and about 42 mm, such as about 39 mm.

With reference to FIG. 2B, different from the planar configuration of the first side member 204, the second side member 206 includes a curved portion with a convex surface facing the first side member 204 such that the first side member 204 and the second side member 206 diverge from the base member 202. Thus, the distance between the first side member 204 and the second side member 206 at a vertical position above the base member 202 is greater than the distance between the first side member 204 and the second side member 206 at the base member 202 or the width of the base member 202.

The curved portion also forms a concave surface that substantially conforms to the curvature or contour of the inferior or lower turbinate 106. As compared to the conventional Doyle splint which contacts the surfaces of one or more turbinates with the convex surface of its half pipe, the concave surface of the second side member 206 of the present technology distributes the pressure imparted on the inferior or lower turbinate 106 to a smaller surface area in a uniform manner, and thus is more comfortable for the patient.

The septum support 200 is configured so that the first side member 204 and the second side member 206 are biased away from one another. The biased configuration results in the first side member 204 contacting and/or pressing against the septum and the second side member 206 contacting and/or pressing against the inferior or lower turbinate 106. In some embodiments, the septum support 200 is resilient such that the first side member 204 and the second side member 206 can be pinched together to reduce the distance between the first side member 204 and the second side member 206 to facilitate insertion of the septum support 200 into the nasal cavity. Once placed into the nasal cavity, the resiliency of the septum support 200 restores the distance between the first side member 204 and the second side member 206 at least to the extent such that the first side member 204 and the second side member 206 are biased away from each other to contact and/or press against the septum and the inferior or lower turbinate 106, respectively. Such biased configuration allows the first side member 204 to apply pressure to one side of the septum. When operating in pair, the left and right septum supports apply pressure to both sides of the septum, and the planar configuration of the respective first side members maintain a straight and/or upright position of the septum.

To achieve the desired resiliency and the biased configuration, in some embodiments, the first side member 204 and/or the second side member 206 are made of a resilient and/or flexible material, such as a shape memory material including a shape memory alloy, a superelastic material, e.g., a superelastic alloy, an elastic material, a polymeric material, or other resilient and/or flexible material. In some embodiments, the first side member 204 and/or the second side member 206 are made of silicone. The resiliency of the material forming the first side member 204 and/or the second side member 206 restores the shapes of and/or distance between the first side member 204 and the second side member 206, and biases the first side member 204 and/or the second side member 206 from each other when inserted into the nasal cavity.

In some embodiments, at least one of the first side member 204 and/or the second side member 206 includes a reinforcing member, such as reinforcing member 234 and/or reinforcing member 236 shown in FIG. 2C, which may be at least partially embedded in the first side member 204 and/or the second side member 206 and may be configured to bias the first side member 204 and the second side member 206 apart.

In some embodiments, the base member 202 is made of the same material as the first side member 204 and/or the second side member 206, while in other embodiments, the base member 202 is made of a material different from that used for making the first side member 204 and/or the second side member 206. In some embodiments, the base member 202 is made of a resilient and/or flexible material, such as a shape memory material including a shape memory alloy, a superelastic material, e.g., a superelastic alloy, an elastic material, a polymeric material, or other resilient and/or flexible material. In some embodiments, the base member 202 is made of silicone.

In some embodiments, the base member 202 is more rigid than the first side member 204 and the second side member 206 so as to maintain the distance between the first side member 204 and the second side member 206 and support the septum support 200 in a substantially upright position inside the nasal cavity. Yet, the base member 202 may still be flexible enough to allow bending and/or folding to facilitate insertion of the septum support 200 into the nasal cavity. In some embodiments, the base member 202 may be made of a material that is more rigid than the material used for making the first side member 204 and/or the second side member 206. In some embodiments, the base member 202 may include a greater thickness than that of the first side member 204 and/or the second side member 206 to achieve desired rigidity. For example, the first side member 204 and/or the second side member 206 may include a thickness that is less than or about 1 mm, less than or about 0.8 mm, less than or about 0.6 mm, or less, whereas the base member 202 may include a thickness that is greater than or about 1 mm, greater than or about 1.2 mm, greater than or about 1.4 mm, or greater.

In some embodiments, the base member 202 is less rigid than the first side member 204 and/or the second side member 206 such that the base member 202 can be easily bent and/or folded to facilitate insertion of the septum support 200 into the nasal cavity. Once the septum support 200 is placed inside the nasal cavity, a reinforcing member may be positioned between the first side member 204 and the second side member 206 and rest on the base member 202 to maintain the planar configuration of the base member 202 inside the nasal cavity.

In some embodiments, whether the base member 202 is less rigid or more rigid, or has a similar rigidity to that of the first side member 204 and/or the second side member 206, a reinforcing member may be nonetheless included. In some embodiments, a reinforcing member, such as reinforcing member 232 shown in FIG. 2C, may be embedded in the base member 202. Although FIG. 2C illustrates three separate, individual reinforcing members 232, 234, 236, the reinforcing members 232, 234, 236 may be formed as one single, continuous piece embedded in the base member 202, the first side member 204, and the second side member 206.

In some embodiments, a reinforcing member, such as reinforcing member 240 shown in FIG. 2D, may be placed between the first side member 204 and the second side member 206 to maintain the planar configuration of the base member 202 inside the nasal cavity. In some embodiments, the reinforcing member 240 may also rest on the base member 202, which may support the reinforcing member 240.

In some embodiments, the reinforcing member 240 may be positioned between the first side member 204 and the second side member 206 and above the base member 202, such as shown in FIG. 2E. Positioning the reinforcing member 240 above the base member 202 and between the first side member 204 and the second side member 206 biases the first side member 204 and the second side member 206 away from each other. The first side member 204 and the second side member 206 may include grooves, such as groove 242 shown in the inset of FIG. 2E, that are configured to engage the reinforcing member 240. This may allow the reinforcing member 240 to be placed at various heights (as indicated in dashed lines in FIG. 2E) to adjust the distance between the first side member 204 and the second side member 206 to better fit the septum support to different patients. In some embodiments, a reinforcing member of a common size may be placed at various heights, while in other embodiments, reinforcing members of various sizes may be selected and placed at various heights to accommodate different patients or for other considerations.

The reinforcing member 240 may be made of the same material as the base member 202, the first side member 204, and/or the second side member 206, or may be made of a different material. In some embodiments, the reinforcing member 240 is made of a resilient and/or flexible material, such as a shape memory material including a shape memory alloy, a superelastic material, e.g., a superelastic alloy, an elastic material, a polymeric material, or other resilient and/or flexible material. In some embodiments, the reinforcing member 240 is made of silicone.

Although FIGS. 2A and 2B illustrate that the base member 202, the first side member 204, and the second side member 206 are each made of a continuous piece of material, one or more of the base member 202, the first side member 204, and/or the second side member 206 may be made of a mesh or wire material. The septum support 200 may be made using any suitable manufacturing methods or techniques depending on the material(s) used to form the septum support 200. For example, the septum support 200 may be molded as one integral piece into the desired shape and configuration. In some embodiments, the molding material may be molded around one or more reinforcing members, such as reinforcing members 232, 234, 236 described herein, to form the septum support 200. In some embodiments, the septum support 200 may be made by scoring and/or folding or bending a piece of resilient material into the desired shape and configuration.

FIG. 2F illustrates a side or lateral view of a septum support 250 that is configured for maintaining a position of a septum of a patient. FIG. 2G illustrates a front or anterior view of the septum support 250. The septum support 250 illustrated in FIGS. 2F and 2G is a right septum support that is configured to be placed or inserted into the right side of the nasal cavity. As will be described in more detail below, in operation, the right septum support operates together with a left septum support that is constructed similarly to the right septum support but is configured to be placed or inserted into the left side of the nasal cavity to maintain the septum in an upright or straight position in a postoperative patient. The septum support 250 of FIGS. 2F and 2G is similar to the septum support 200 of FIGS. 2A and 2B and, thus, some of the description of the septum support 250 will be omitted for brevity. It should be realized, however, that the description of the septum support 200 of FIGS. 2A and 2B is equally applicable to the septum support 250 of FIGS. 2F and 2G.

As shown in FIGS. 2F and 2G, the septum support 250 includes a base member 252, a first side member 254 extending upward away from a first edge of the base member 252, and a second side member 256 extending upward away from a second edge of the base member 252. The base member 252, the first side member 254, and the second side member 256 collectively define a channel or a trough with uneven sides. The channel or trough may be U-shaped or any other shaped that defines or forms an opening between the first side member 254 and the second side member 256. The more open design of the septum support 250 provides a breathing passage for the patient that is less likely to be blocked in comparison with a Doyle splint and any accumulation can be more easily rinsed out with saline or other solutions.

The base member 252 is substantially planar and is configured for positioning on a bottom or floor of the nasal cavity of the patient. The first and second side members 254, 256 that extend upward from the base member 252 are designed to maintain the orientation and/or position of the septum support 250 in the nasal cavity. In some embodiments, the width of the base member 252 from the first edge to the second edge ranges between about 2 mm and about 8 mm, between about 3 mm and about 6 mm, such as about 4 mm. The width of the base member 252 may be consistent throughout its longitudinal extension. The first side member 254 and the second side member 256 typically have different longitudinal lengths. Specifically, as illustrated in FIG. 2F the second side member 256 has a longitudinal length that is substantially shorter than the longitudinal length of the first side member 254. In such embodiments, the second side member 256 may be positioned roughly centrally between a proximal edge 266 and a distal edge 268 of the first side member 254. The longitudinal length of the first side member 254 is typically coextensive with, or equal to, the longitudinal dimension of the base member 252. In some embodiments, the second side member 256 may also have a longitudinal length that is substantially the same, or coextensive with, the length of the base member 252.

The longitudinal length of the first side member 254 is defined between its proximal edge 266 and distal edge 268. The length of the first side member 204 may substantially correspond to the length of the septum such that the first side member 204 supports substantially the entire septum along the longitudinal extension of the septum. In some embodiments, the length of the first side member 254 ranges between about 65 mm and about 75 mm or between about 65 mm and about 70 mm, such as about 68 mm. A height of the first side member 254 is defined between an upper edge 272 and the base member 252. The height of the first side member 254 may range between about 16 mm and about 26 mm, between about 18 mm and about 24 mm, or between about 20 mm and about 24 mm, such as about 22 mm. The upper edge 272 may be substantially straight while the proximal and distal edges 266, 268 are curved so as to form tapered proximal and distal ends of the septum support 250. The curved ends of the septum support 250 may facilitate insertion and removal of the septum support 250 from the nasal cavity.

The first side member 254 may include an aperture or suture hole 260 that is positioned near the proximal edge 266 and/or distal edge 268. The suture hole(s) 260 allow the first side member 254 to be sutured to the septum near the columella. Alternatively, or additionally, the first side member 254 may be adhered to the septum using adhesive or glue. In a specific embodiment, the septum support 250 includes a pair of suture holes 260 that are positioned on opposing sides of the septum support 250 near the proximal edge 266 and the distal edge 268. The suture holes 260 may be vertically aligned about the septum support 250 and may be spaced between 60 mm and 64 mm apart on center, and more commonly about 62 mm apart on center.

When the septum support 250 is inserted into the right side of the nasal cavity, the first side member 254 is configured to contact the right side of the septum, and the second side member 256 is configured to contact a turbinate, such as the inferior or lower turbinate 106, on the right lateral wall of the nasal cavity. The first side member 254 is substantially planar so as to maintain the septum in a substantially upright or straight position. The second side member 256 is configured to contact the lower turbinate 106 on the right lateral wall of the nasal cavity while limiting or avoiding contact with the middle turbinate 104. To avoid contact with the middle turbinate 104, a height of the second side member 256 as defined by an upper edge 274 of the second side member 256 and the base member 252 is substantially shorter than the height of the first side member. In some instances, the height of the second side member 256 may be between about 3 mm and about 13 mm, or between about 4 mm and about 8 mm, such as about 5 mm. Additionally, the second side member 256 includes a length as defined by a proximal edge 276 and a distal edge 278 that is less than the length of the first side member 254 such that the second side member 256 does not extend as far into the nasal cavity as the first side member 254. This may further avoid or minimize contact with the middle turbinate 104 and/or limit or prevent blockage or irritation of the sinuses. The length of the second side member 256 typically ranges between about 30 mm and about 50 mm or between about 35 mm and about 45 mm, such as about 40 mm.

As illustrated in FIG. 2G, the second side member 256 includes a curved portion with a convex surface facing the first side member 254 such that the first side member 254 and the second side member 256 diverge from the base member 252. Thus, the distance between the first side member 254 and the second side member 256 at a vertical position above the base member 252 is greater than the distance between the first side member 254 and the second side member 256 at the base member 252. The curved portion forms a concave surface that may substantially conform to the curvature or contour of the inferior or lower turbinate 106. In some instances, the second side member 256 may curve outward so that an outer edge of the second side member 256 is positioned between about 0.5 mm and about 2 mm, and more about commonly 1 mm, away from the outer edge of the base member 252.

As further illustrated in FIG. 2G, a distal end of the second side member 256 includes an outward extending rim or lip 282 (hereinafter rim 282). In this context, the term “outward” is used in reference to the U-shaped channel or trough that is defined by the first side member 254 and the second side member 256. The rim 282 extends outward from the U-shaped channel or trough rather than extending into or toward the U-shaped channel or trough. Thus, the rim 282 extends from the second side member 256 in a direction away from the first side member 154. The rim 282 has a thickness that is roughly equivalent to a thickness of the second side member 256 and/or first side member 254. Specifically, the rim 282 may have a thickness of between 0.5 mm and 2 mm, and more commonly about 1 mm, which corresponds to the thickness of the first side member 254 and/or second side member 256.

The rim 282 may extend outward from the second side member 256 by between 0.5 and 3 mm, more commonly between 0.5 mm and 2 mm, and most commonly about 1 mm. The rim 282 typically extends along the entire length of the second side member 256, although in some instances the rim 282 may extend less than the entire length of the second side member 256. For example, the length of the rim 282 may be between 30 mm and 45 mm, and more commonly between 35 mm and 45 mm. In a specific embodiment, the length of the rim 282 is roughly 40 mm.

When the septum support 250 is positioned within the nasal cavity, the rim 282 directly contacts the lower turbinate 106. The rim 282 may contact the lower turbinate 106 to form or maintain an air passage through the nasal cavity. The rim 282 may further facilitate insertion and/or removal of the septum support from the nasal cavity.

The septum support 250 is configured so that the first side member 254 and the second side member 256 are biased away from one another as previously described, which helps maintain the air passage through the nasal cavity. The septum support 250 is also resilient such that the first side member 254 and the second side member 256 can be pinched together to facilitate insertion into the nasal cavity. To achieve the desired resiliency and the biased configuration, in some embodiments, the first side member 254 and/or the second side member 256 are made of a resilient and/or flexible material, such as a shape memory material including a shape memory alloy, a superelastic material, e.g., a superelastic alloy, an elastic material, a polymeric material, or other resilient and/or flexible material. In some embodiments, the first side member 254 and/or the second side member 256 are made of silicone. The resiliency of the material forming the first side member 254 and/or the second side member 256 restores a shape of, and/or distance between, the first side member 254 and the second side member 256, and biases the first side member 254 and/or the second side member 256 away from each other when inserted into the nasal cavity.

In some embodiments, the septum support 250 may include one or more additional rims or lips. For example, the septum support 250 may include an upper rim or lip 286 (hereinafter upper rim 286) that extends outward from the first side support 254. In this context, the term “outward” is used to mean that the upper rim 286 extends in the same general direction as the rim 282 of the second side member 256. Specifically, the upper rim 286 extends from the first side member 254 in a direction away from the septum. The upper rim 286 typically has a length that is shorter than the length of the first side member 254 as measured between the proximal edge 266 and the distal edge 268. In some instances, the length of the upper rim 286 may be roughly equal to the length of the rim 282, while in other instances the upper rim 286 may be longer or shorter than the rim 282. Specifically, the length of the upper rim 286 may be between 30 mm and 45 mm, and more commonly between 35 mm and 45 mm. In a specific embodiment, the length of the upper rim 286 is roughly 40 mm.

The upper rim 286 may extend outward from the first side member 254 by between 0.5 mm and 2 mm and more commonly about 1 mm. The thickness of the upper rim 286 may similarly be between 0.5 mm and 2 mm, and more commonly about 1 mm. An upper surface of the upper rim 286 may be positioned between 4 mm and 6 mm from the upper edge 272. In a specific embodiment, the upper surface of the upper rim 286 may be positioned approximately 5 mm from the upper edge 272. The upper rim 286 may reinforce the septum support 250 to prevent or minimize unwanted bending, twisting, or folding of the septum support 250 within the nasal cavity.

The septum support 250 may also, or alternatively, include a middle rim or lip 284 (hereinafter middle rim 284) that extends outward from the first side support 254. In this context, the term “outward” is used to mean that the middle rim 284 extends in the same general direction as the upper rim 286. The middle rim 284 typically has a length that is shorter than the length of the first side member 254 as measured between the proximal edge 266 and the distal edge 268, but is longer than the rim 282 and/or upper rim 286. In some instances, however, the middle rim 284 may be shorter than the rim 282 and/or upper rim 286. In some instances, the length of the middle rim 284 is between 50 mm and 60 mm, and more commonly between 54 mm and 58 mm. In a specific embodiment, the length of the middle rim 284 is roughly 56 mm.

The middle rim 284 extends outward from the first side member 254 by between 0.5 mm and 2 mm and more commonly about 1 mm. In some instances, the middle rim 284 may extend outward from the first side member 254 farther than the upper rim 286 extends from the first side member 254. The thickness of the middle rim 284 may similarly be between 0.5 mm and 2 mm, and more commonly about 1 mm. A lower surface of the middle rim 284 may be positioned between 10 mm and 14 mm from the upper edge 272. In a specific embodiment, the lower surface of the middle rim 284 may be positioned approximately 12 mm from the upper edge 272. The middle rim 284 may reinforce the septum support 250 to prevent or minimize unwanted bending, twisting, or folding of the septum support 250 within the nasal cavity. The use of the middle rim 284 and upper rim 286 may greatly reinforce the septum support 250, although only one of the rims, 284 or 286, may be used in some embodiments.

In some embodiments, the second side member of the nasal splint or septum support may flex inward so that the second side member does press on the nose in an irritating and uncomfortable manner. In such embodiments, the second side member may be fixedly attached or secured to the base or lower edge of the septum support and may extend upward from the base or lower edge a distance that is sufficient to apply pressure to the inferior turbinate while also being able to fit or slide under the middle turbinate. For example, the second side support may be roughly 6-8 mm in height from the base of the nasal splint. Pressure against the inferior turbinate is sufficient to support the septum. The upper end of the second side member curves inward so that it is easily compressible and does not dig or press into the nose. The upper end of the second side member is not attached to the first side member of the nasal splint. Because the upper end of the second side member does not connect to the first side member, it is easy to squeeze the second side member inward toward the first side member and is more conformable to the shape and size of the nose. In addition, the lower height of the second side member minimizes the amount of mucus/clot collection in the nasal airway, making removal of the mucus/clot easier.

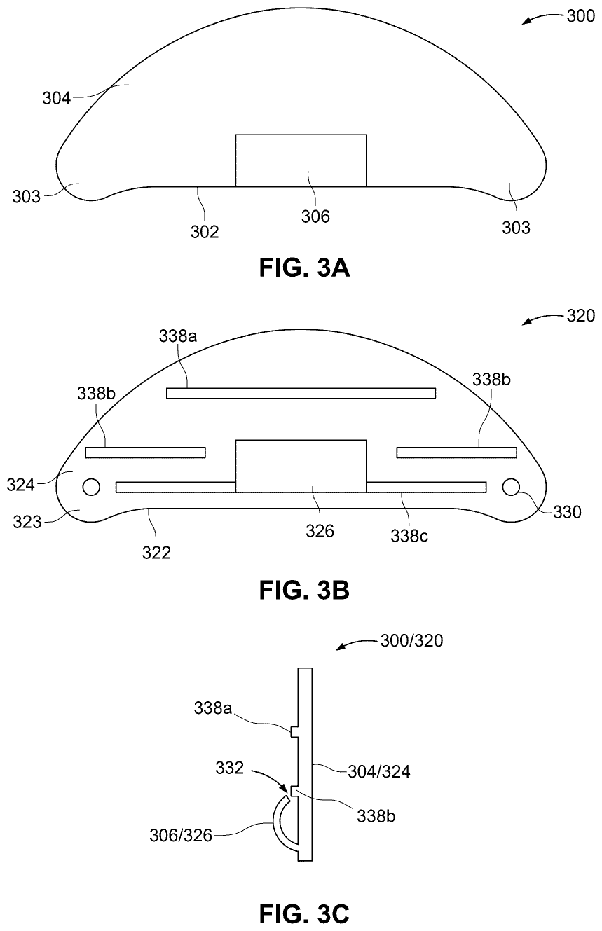

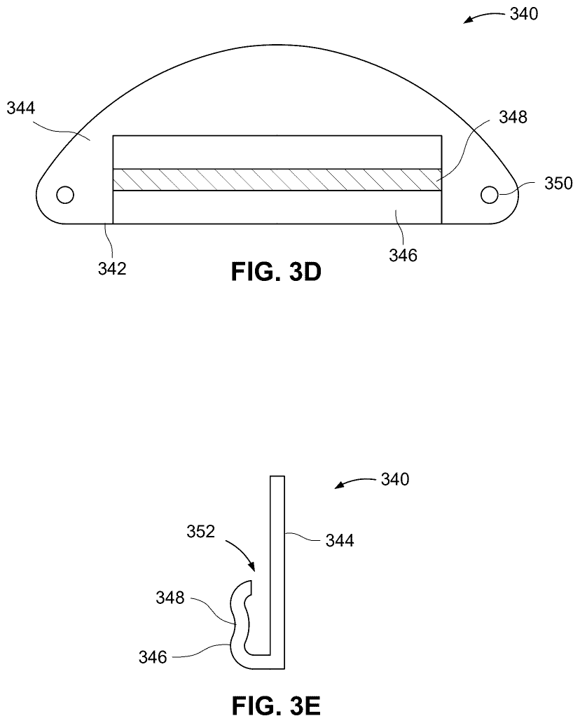

Referring to FIG. 3A, illustrated is a nasal splint or septum support 300 (hereinafter nasal splint 300) that includes a base 302, a first side member 304, and a second side member 306. The first side member 304 may also be referred to as a septum support or septal plate or lamella (hereinafter septum support 304). Similarly, the second side member 306 may be referred to as a side support or turbinate brace (hereinafter side support 306). It should be realized that these terms can be used interchangeably herein to describe any of the various embodiments.

The base 302 is generally elongated and in some instances may include a straight central section with one or more opposing ends 303 that curve downward relative to the central portion. FIGS. 3A-3B illustrate embodiments in which the nasal splint includes a straight central section and opposing ends 303 that curve downward such that the nasal splint has a slight boomerang shape. The curvature may be designed to conform to the anatomy of the nasal cavity and to minimize patient discomfort. In some embodiments, the opposing ends 303 may curve downward about 1 mm from the central portion of the base 302. FIG. 3D illustrates an embodiment in which the nasal splint includes a relatively straight base without opposing ends that curve downward. Any of the nasal splint embodiments described herein may include a straight base with or without one or more opposing curved ends as desired.

The septum support 304 is positioned on a first side of the base 302 and extends or projects upward from the base 302. The septum support 304 is configured to contact and engage the septum within the nasal cavity, thereby providing direct stabilization. The septum support 304 may have a planar or contoured surface that is adapted to the septal anatomy. The septum support 304 typically has a length between opposing sides that ranges between about 65 mm and about 75 mm, or between about 65 mm and about 70 mm, such as about 68 mm. In instances where the opposing ends 303 curve downward, the straight central portion may be between 45 mm and 55 mm, such as about 50 mm. The septum support 304 may have a height defined between the base 302 and an uppermost edge that is sufficient to support substantially the entire septum. In some embodiments, the height of the septum support 304 may range between about 15 mm and about 25 mm, between about 17 mm and about 25 mm, such as about 23 mm.

The side support 306 is positioned on the opposite side of the base 302. The side support 306 also extends or projects upward from the base 302, but a height of the side support 306 as measured from the base 302 to an uppermost edge of the side support 306 is typically less than half the height of the septum support 304. The side support 306 is designed to contact the inferior turbinate of the nasal cavity to reinforce the septum support 304 by providing lateral stability to the septum support 304. The side support 306 is shaped, sized, and arranged so that the side support 306 does not contact the middle turbinate within the nasal cavity. Rather, the side support 306 is designed to slide under the middle turbinate or otherwise be positioned under the middle turbinate. For example, the side support 306 may extend upward from the base 302 in the range of approximately 6 mm to 8 mm, which ensure that the side support 306 does not contact and engage the middle turbinate. This design stands in contrast to conventional splints that are often designed to engage the middle turbinate or that incidentally contact the middle turbinate. In sinus surgery, the middle turbinate is often mobilized, which makes it easier for the middle turbinate to get displaced laterally and form adhesions to the lateral nasal wall. This concern is obviated with the nasal splint described herein. In some embodiments, the vertical height of the side support 306 may be constant or variable along its length, but is typically limited to a height of less than about 8 mm, such as within a range of 6-8 mm. The smaller height of the side support 306 in comparison with conventional splints may also minimize the amount of clot that is collected within the splint, thereby increasing or improving the breathing passage. The length of the side support 306 is typically substantially shorter than the length of the septum support 304. For example, the side support 306 may have a length that is less than one third the length of the septum support 304. In some instances, the side support 306 may have a length between opposing sides ranging from about 15 mm to about 25 mm, such as about 20 mm. Alternatively, in some embodiments, the side support 306 may have a length up to about 30 mm or about 50 mm. Shorter side supports 306 may minimize the collection of mucous and/or clot within a channel or trough (see FIG. 3C). The bottom end or edge of the side support 306 may be flush or aligned with the bottom end or edge of the base 302.

In the embodiments described herein, the side support may be positioned or located roughly central about a longitudinal length of the nasal splint. In other embodiments, however, the side support may be positioned closer to one end or edge of the nasal splint. Similarly, in some embodiments, the shape and arrangement of the nasal splint may be mirrored about a plane positioned along a longitudinal center of the nasal splint and orthogonal to the nasal splint. In other embodiments, the shape and/or arrangement of one or more features may not be mirrored about such a plane.

As illustrated in FIG. 3C, the side support 306/326 has a generally arcuate or semicircular profile such that its lower end curves outward away from the septum support 304/324 and its upper end curves inward toward the septum support 304/324. The lower end of the side support 304/324 is securely attached to either the base 302 or the septum support 304/324, while the upper end is and/or remains unattached to the septum support 304/324. Stated differently, the upper end of the septum support 304/324 is not connected, and is commonly never fixedly connected, to the septum support 304/324. This configuration allows the side support to flexibly engage the inferior turbinate while maintaining the necessary biasing force against the septum. The base 302, septum support 304, and side support 306 collectively define a channel or trough 332, which may have a curved shape, such as a C-shape, U-shape, or semicircular shape. The curved shape of the side support 306 and/or channel or trough 332 may be configured to accommodate the anatomical contours of the nasal cavity. The curved shaped of the channel or trough 332 may also enhance the ability of the nasal splint to stabilize the septum and adjacent tissues. The side support 306/326 may extend laterally outward from the septum support 304/324 by between 3 mm and 5 mm, such as about 4 mm.

Referring to FIG. 3B, an alternative nasal splint 320 is illustrated. The nasal splint 320 is substantially identical to the nasal splint 300 of FIG. 3A except in the inclusion of one or more features and/or the position of one or more features. For example, the nasal splint 320 includes one or more reinforcing ribs 338a-c that extend longitudinally along its length. These ribs 338a-c protrude laterally outward from the surface of the septum support 324, preferably in the direction of the side support 326. The ribs 338a-c enhance the rigidity of the septum support 324 thereby resisting deformation of the septum support 324 and nasal splint 320. In the illustrated embodiment, the nasal splint 320 includes a superior rib 338a that is positioned near an upper edge of the septum support 324. The superior rib 338a may have a length of between 35 mm and 55 mm, such as about 40 mm. The nasal splint 320 also includes at least one middle rib 338b and often two middle ribs 338 that are positioned on opposing sides of the side support 326. The middle rib(s) 338b is typically aligned with an upper edge of the side support 326 as illustrated in FIG. 3C. Alignment of the middle rib(s) 338b with the upper edge of the side support 326 may reinforce the septum support 324 and nasal splint 320 at this interface. The middle rib(s) 338b is slightly offset laterally from the side support 326 so that the side support 326 does not contact or engage the middle rib(s) 338b. The middle rib(s) 338b may have a length of between about 10 mm and 20 mm, such as about 15 mm.

In some instances, the nasal splint 320 also includes an inferior rib 338c. In such instances, the inferior rib 338c typically extends along a majority of the length of the base 322, such as along the straight central portion between the opposing curved ends 323. The inferior rib 338c may have a length of between about 35 mm and 55 mm, such as about 50 mm. The inferior rib 338c may be aligned with the base 322 so that it is flush with a bottom end or edge of the base 322, or the inferior rib 338c may be positioned offset from the base 322, such as the position of the inferior rib 338c in FIG. 3B. In the latter embodiment, the inferior rib 338c may be positioned 1 mm to 5 mm offset from the base 322. In some instances, the bottom end of the side support 326 may also be positioned between 1 mm and 3 mm offset from the base 322, although the bottom end of the side support 326 is commonly aligned with the base 322.

In certain embodiments, one or both of the opposing ends 323 of the base 322 are provided with an aperture 330. The aperture 330 enables the nasal splint 320 to be anchored to the septum using a suture. This feature may help ensure long-term stability of a position of the splint 320 within the nasal cavity.

Referring to FIGS. 3D-E, illustrated is an alternative embodiment of a nasal splint 340. The nasal splint 340 is substantially identical to the nasal splints described herein except in the inclusion of one or more features and/or the position of one or more features. For example, the nasal splint 340 a base 342, a septum support, septal plate, or septal lamella 344 (hereinafter septal lamella 344), and a side support or turbinate brace 346 (hereinafter turbinate brace 346). The septal lamella is positioned on a first side of the base 342 and extends upward therefrom as described herein. Similarly, the turbinate brace 346 is positioned on a second side of the base 342 and extends upward therefrom as described herein. The geometries, sizes, shapes, and/or orientations of these features may be as described herein, especially in relation to FIGS. 3A-C. The turbinate brace 346 is illustrated as being substantially longer than those illustrated in FIGS. 3A-B, but the turbinate brace 346 may have a shorter length as desired.

A lower end of the turbinate brace 346 is securely connected to the base 342 or to the septal lamella 344 while an upper end of the turbinate brace 346 is not attached to the septal lamella 344. The turbinate brace 346 has an arcuate or semicircular profile such that the lower end of the turbinate brace curves outward away from the septal pad and the upper end of the turbinate brace curves inward toward the septal pad. In the illustrated embodiment, the turbinate brace 346 has a S-shaped profile (see FIG. 3E). The S-shaped profile may be due to the inclusion or formation of a groove 348 in an outer surface or wall of the turbinate brace 346. The groove 348 may extend along an entire longitudinal length of the turbinate brace 346. The groove 348 is designed to facilitate secure manipulation or handling. For example, the groove 348 may enable the splint 340 to be easily and securely grasped with a blade or jaw of a forceps, or similar instrument, to enable insertion of the splint 340 within the nasal cavity or removal of the splint 340 therefrom. The groove 348 may be shaped and sized to correspond to the blade or jaw of the forceps, such as by being between 1 mm and 3 mm wide as measured between the base 342 and upper edge of the septal lamella 344. In a specific embodiment, the groove 348 may be roughly about 2 mm wide. The groove 348 allows the forceps to grip and squeeze the splint 340 to keep it in position. In some instances, a notch at the distal end of the turbinate brace 346 may contact the surface of the septal lamella 344 to provide reinforcement and/or prevent closure of the open channel.

The turbinate brace 346 may extend laterally outward from the surface of the septal lamella 344 by between 3 mm and 5 mm, such as about 4 mm. While not illustrated, the septal lamella 344 may include one or more reinforcing ribs that extend longitudinally along the septal lamella and laterally outward therefrom as described herein. The nasal splint 340 may also include opposing ends that curve downward as described.

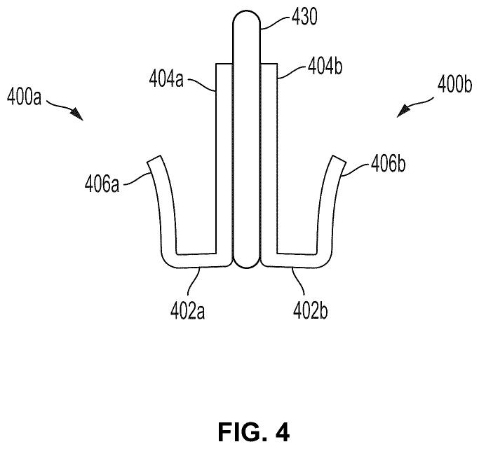

FIG. 4 illustrates a front or anterior view of a pair of septum supports or nasal splints that engage a septum to support the septum. The septum supports or nasal splints are representative of any of the septum supports or nasal splints described herein. FIG. 4 includes a right septum support 400a and a left septum support 400b that operate together to maintain an upright position of the septum 430. The right septum support 400a essentially is a mirror image of the left septum support 400b with the septum 430 being the plane of reflection. The left septum support 400b and the right septum support 400a each include a base member, 402b & 402a, that is configured to be placed on a bottom or floor of the respective side of the nasal cavity. The left septum support 400b and the right septum support 400a each include a septum support, 404b & 404a, configured to be placed in contact with respective sides of the septum 430. The left septum support 400b and the right septum support 400a each include a side support, 406b & 406a, configured to be placed in contact with the inferior or lower turbinate (not shown in FIG. 4) on respective lateral walls of the nasal cavity. The biased configuration of the respective septum support 404a, 404b and the side support 406a, 406b allows pressure to be applied to either side of the septum 430 such that the septum 430 is maintained in an upright and/or straight position as the septum 430 heals after the operational procedure.

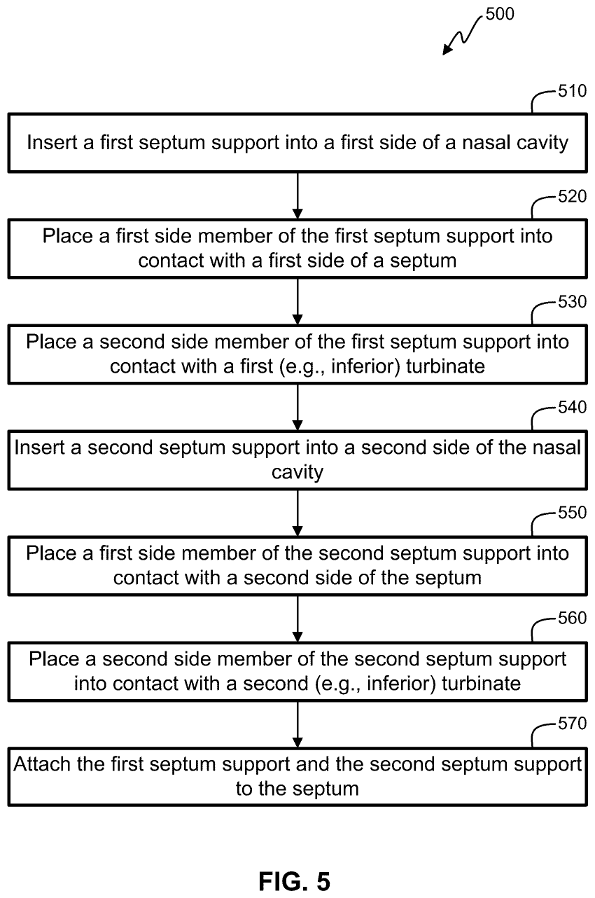

FIG. 5 is a simplified flow chart 500 illustrating a method of using the septum supports described herein to help retain a breathing passage and/or to maintain the position of the septum for a patient.

At block 510, a first septum support is inserted into one side of a nasal cavity. The first septum support may be a left or right septum support as described herein. The first septum support is resilient or flexible such that the first septum support may be pinched, bended, or folded to facilitate the insertion of the first septum support. In some instances, pressure may be applied to a first side member and a second side member of the first septum support to reduce the distance between the first side member and the second side member to facilitate insertion of the first septum support into the one side of the nasal cavity. Once the first septum support is inserted into the nasal cavity, the pressure may be released from the first side member and the second side member to restore at least a portion of the distance between the first side member and the second side member.

At block 520, the position of the first septum support may be adjusted so as to place the first side member into contact with one side of the septum. At block 530, the second side member is placed into contact with a turbinate, such as the inferior or lower turbinate.

At block 540, a second septum support is inserted into the other side of the nasal cavity. The second septum is a mirror image of the first septum support. Similar to the first septum support, the second septum support is resilient or flexible such that the second septum support may be pinched, bent, or folded to facilitate the insertion of the second septum support into the nasal cavity. For example, pressure may be applied to a first side member and a second side member of the second septum support to reduce the distance between the first side member and the second side member to facilitate insertion of the second septum support into the nasal cavity. Once the second septum support is inserted into the nasal cavity, the pressure may be released from the first side member and the second side member to restore at least a portion of the distance between the first side member and the second side member.

At block 550, the position of the second septum support may be adjusted so as to place the first side member of the second septum support into contact with the other side of the septum. At block 560, the second side member is placed into contact with another turbinate, such as the other inferior or lower turbinate.

As discussed above, the first and second side members are in a biased configuration. Such configuration allows pressure to be applied to either side the septum once the first and second septum supports are placed inside the nasal cavity, maintaining the septum in an upright or straight position.

At block 570, the first septum support and the second septum support are attached to the septum so as to maintain the position of each septum support relative to the septum. The respective first members of the first and second septum supports may each include a suture hole, and a common suture may be applied through both suture holes to secure the first and second septum supports in place. Alternatively, the first and second septum supports may be secured in place via gluing, adhering, or another suitable attaching mechanism.

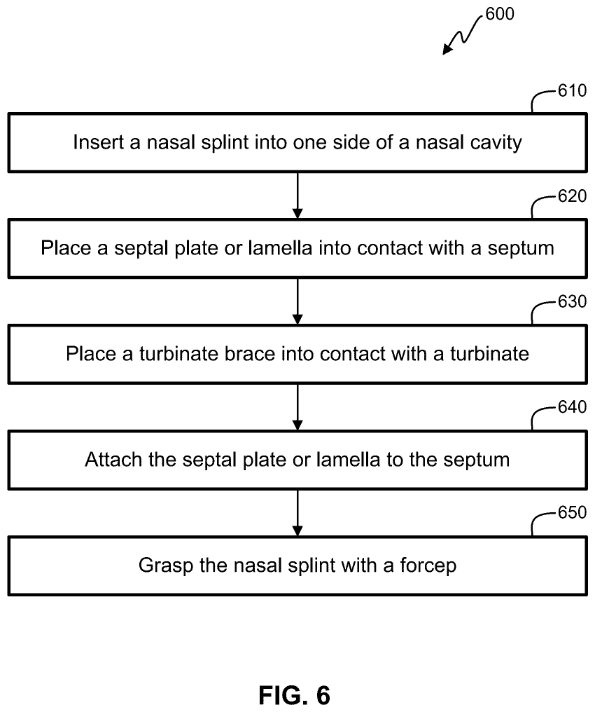

FIG. 6 is a simplified flow chart 600 illustrating a method of using the septum supports or nasal splints described herein to help retain a breathing passage and/or to maintain the position of the septum for a patient.

At block 610, a nasal splint is inserted into one side of a nasal cavity. As described herein, the nasal splint may include a base, a septal plate or lamella, and a turbinate brace, in which a lower end of the turbinate brace is securely connected to the base or to the septal plate or lamella and an upper end of the turbinate brace is not attached to the septal plate or lamella. At block 620, the septal plate or lamella is placed into contact with the septum. At block 630, the turbinate brace is placed into contact with a turbinate of the nasal cavity so that the turbinate brace engages the turbinate. The septal plate or lamella and the turbinate brace are biased away from one another such that pressure is applied to the septum by the septal plate or lamella. At block 640, the method optionally includes attaching the septal plate or lamella to the septum to maintain a position of the nasal splint relative to the septum. The septal plate or lamella may be attached to the septum via one or more sutures as described herein. At block 650, the method optionally includes grasping the nasal splint by positioning a blade or jaw of a forcep within a groove that extends along a longitudinal length of the turbinate brace.

The following embodiments described are directed to a nasal splint that is positionable within the nose to help maintain the operated septum in a midline position.

While several embodiments and arrangements of various components are described herein, it should be understood that the various components and/or combination of components described in the various embodiments may be modified, rearranged, changed, adjusted, and the like. For example, the arrangement of components in any of the described embodiments may be adjusted or rearranged and/or the various described components may be employed in any of the embodiments in which they are not currently described or employed. As such, it should be realized that the various embodiments are not limited to the specific arrangement and/or component structures described herein.

In addition, it is to be understood that any workable combination of the features and elements disclosed herein is also considered to be disclosed. Additionally, any time a feature is not discussed with regard in an embodiment in this disclosure, a person of skill in the art is hereby put on notice that some embodiments of the invention may implicitly and specifically exclude such features, thereby providing support for negative claim limitations.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the device” includes reference to one or more devices and equivalents thereof known to those skilled in the art, and so forth.

Also, the words “comprise,” “comprising.” “include,” “including” and “includes” when used in this specification and in the following claims are intended to specify the presence of stated features, integers, components, or steps, but they do not preclude the presence or addition of one or more other features, integers, components, steps, acts, or groups.

What is claimed is:

1. A nasal splint configured for insertion into a patient's nasal cavity to stabilize a septum, comprising:
a base;
a septum support situated on a first side of the base and extending upward therefrom, the septum support being configured to engage the septum within the nasal cavity; and
a side support located on a second side of the base and extending upward therefrom, the side support being configured to contact a turbinate of the nasal cavity so as to reinforce the septum support;
wherein a lower end of the side support is securely connected to either the base or septum support;
wherein an upper end of the side support is unattached to the septum support; and
wherein the side support has an arcuate or semicircular profile such that its lower end curves outward away from the septum support and its upper end curves inward toward the septum support.

2. The nasal splint of claim 1, wherein the side support extends upward from the base to a height that is less than half a height of the septum support measured from the base such that the side support does not contact a middle turbinate of the nasal cavity.

3. The nasal splint of claim 2, wherein the side support extends upward from the base between 6 and 8 mm, and wherein the septum support extends upward from the base between 20 and 25 mm.

4. The nasal splint of claim 1, wherein the septum support includes one or more reinforcing ribs that extend longitudinally along a length of the septum support and that protrude laterally from a surface of the septum support in a direction toward the side support.

5. The nasal splint of claim 4, wherein at least one reinforcing rib of the one or more reinforcing ribs is aligned with a top edge of the side support.

6. The nasal splint of claim 1, wherein the base, the septum support, and the side support define or form a curved channel or trough.

7. The nasal splint of claim 1, wherein opposing ends of the base curve downward relative to a straight central section.

8. The nasal splint of claim 1, wherein at least one end of the base includes an aperture to allow the nasal splint to be anchored to the septum using a suture.

9. The nasal splint of claim 1, wherein the side support includes a groove that extends along a longitudinal length of the side support.

10. A nasal splint comprising:
a base;
a septal plate or lamella positioned on a first side of the base and extending upward therefrom; and
a turbinate brace positioned on a second side of the base and extending upward therefrom; and
wherein a lower end of the turbinate brace is securely connected to the base or to the septal plate or lamella;
wherein an upper end of the turbinate brace is not attached to the septal plate or lamella; and
wherein the turbinate brace has an arcuate or semicircular profile such that the lower end of the turbinate brace curves outward away from the septal plate or lamella and the upper end of the turbinate brace curves inward toward the septal plate or lamella.

11. The nasal splint of claim 10, wherein the turbinate brace extends upward a height that is less than half a height of the septal plate or lamella measured from the base such that turbinate brace does not contact a middle turbinate.

12. The nasal splint of claim 11, wherein the turbinate brace extends upward from the base between 6 and 8 mm, and wherein the septal plate or lamella extends upward from the base between 20 and 25 mm.

13. The nasal splint of claim 10, wherein the septal plate or lamella includes one or more reinforcing ribs that extend longitudinally along a length of the septal plate or lamella and that protrude laterally from a surface of the septal plate or lamella.

14. The nasal splint of claim 13, wherein at least one reinforcing rib of the one or more reinforcing ribs is aligned with a top edge of the turbinate brace.

15. The nasal splint of claim 10, wherein the base, the septal plate or lamella, and the turbinate brace define or form a curved channel or trough.

16. The nasal splint of claim 10, wherein opposing ends of the base curve downward relative to a straight central section.

17. The nasal splint of claim 10, wherein the turbinate brace includes a groove that extends along a longitudinal length of the turbinate brace.

18. A method for supporting a septum of a nasal cavity, the method comprising:
inserting a nasal splint into one side of the nasal cavity, the nasal splint including:
a base;
a septal plate or lamella; and
a turbinate brace;
wherein a lower end of the turbinate brace is securely connected to the base or to the septal plate or lamella and wherein an upper end of the turbinate brace is not attached to the septal plate or lamella;
placing the septal plate or lamella into contact with the septum; and
placing the turbinate brace into contact with a turbinate of the nasal cavity so that the turbinate brace engages the turbinate;
wherein the septal plate or lamella and the turbinate brace are biased away from one another such that pressure is applied to the septum by the septal plate or lamella.

19. The method of claim 18, further comprising attaching the septal plate or lamella to the septum to maintain a position of the nasal splint relative to the septum.

20. The method of claim 18, further comprising grasping the nasal splint by positioning a blade or jaw of a forcep within a groove that extends along a longitudinal length of the turbinate brace.
